(19) United States
(12) Patent Application Publication
(10) Pub. No.: US 2010/0256038 A1
(43) Pub. Date: Oct. 7, 2010

(54) TH-17 CELLS

(75) Inventor: Adam Curnock, Abingdon (GB)

Correspondence Address: Jonathan P. O''Brien, Ph.D.;Honigman Miller Schwartz & Cohn LLP, 350 East Michigan Avenue, Suite 300, Kalamazoo, MI, 49007, US

(73) Assignee: Vertex Pharmaceuticals Incorporated, Cambridge, MA (US)

(21) Appl. No.: 12/666,040

(22) PCT Filed: Jun. 25, 2008
(86) PCT No.: PCT/US08/07914

§ 371 Date: Mar. 5, 2010

Related U.S. Application Data
(60) Provisional application No. 60/937,113, filed on Jun. 25, 2007.

(51) Int. Cl. A61K 38/02; C12N 5/0783; C12Q 1/02; G01N 33/53; C12Q 1/68; A61K 31/7088; A61K 31/70

(52) U.S. Cl. 514/1.1; 435/372.3; 435/29; 435/7.92; 435/6; 514/44 R; 514/44 A; 514/23

(57) ABSTRACT

The present invention provides methods for stimulating naive T-cells or memory T-cells to produce IL-17, methods for identifying modulators of IL-17 production, and methods for treating IL-17 mediated disorders.

### CLAIM OF PRIORITY

This application claims the benefit of U.S. Provisional application No. 60/937,113 filed on Jun. 25, 2007, which is herein incorporated in its entirety by reference.

### BRIEF DESCRIPTION

The present invention provides methods for stimulating naïve T-cells to produce IL-17, methods for identifying modulators of IL-17 production, and methods for treating IL-17 mediated disorders.

### BACKGROUND

Cytokines are secreted soluble proteins that bind to cell surface receptors, triggering signal transduction pathways that lead to cell activation, proliferation and differentiation. One such cytokine, interleukin-17 (IL-17), originally named CTLA-8, is a T-cell derived pro-inflammatory molecule that stimulates epithelial, endothelial and fibroblastic cells to produce other inflammatory cytokines and chemokines including IL-6, IL-8, G-CSF, PGE2 and MCP-1. (Aggarwal and Gurney, J. Leukoc. Biol. 71:1-8, 2002; Yao et al., Immunity 3:811-21, 1995; Kennedy et al., J. Interferon Cytokine Res. 16:611-17, 1996; Fossiez et al., J Exp Med 183:2593-603, 1996; Linden et al., Eur Respir J 15:973-77, 2000; Cai, et al., Immunol Lett 62, 51-58 (1998); Jovanovic et al., J Immunol 160:3513-21, 1998; and Laan et al., J Immunol 162, 2347-52, 1999).

IL-17 synergizes with other cytokines including TNF-α and IL-1β to further induce chemokine expression (Jovanovic et al., supra, and Chabaud, et al., J Immunol 161:409-14, 1998). Levels of IL-17 are significantly increased in rheumatoid arthritis (RA) synovium (Kotake et al., J Clin Invest 103:1345-52, 1999; and Chabaud et al., Arthritis Rheum 42:963-70, 1999), during allograft rejection (Antonysamy et al., Transplant Proc 31:93 (1999); Antonysamy et al., J Immunol 162:577-84, 1999; Loong, et al. Transplant Proc 32:1773 (2000); and Hsieh, et al., Transpl Int 14:287, 2001), and in other chronic inflammatory diseases including multiple sclerosis (Kurasawa et al., Arthritis Rheum 43:2455-63, 2000) and psoriasis (Albanesi et al., J Invest Dermatol 115, 81-87, 2000, Homey et al., J Immunol 164, 6621-32, 2000).

A new helper T cell subset, TH-17, has recently been identified in the mouse and appears to be responsible for mediating autoimmune inflammation in disease models of multiple sclerosis (MS) and rheumatoid arthritis (RA) (Langrish, et al., J. Exp. Med., 201:233-40, 2005; Weaver, et al., Immunity, 24:677-88, 2006). The pathogenic function of TH-17 cells in autoimmunity is believed to be mediated through their production of the pro-inflammatory cytokine, IL-17. In humans, autoimmune diseases, including RA, MS and psoriasis are associated with increased levels of IL-17, which indicates that an equivalent subset does exist in humans.

### SUMMARY

This invention provides methods for producing TH-17 cells, methods for identifying modulators of IL-17 production in T-cells, and methods for treating IL-17 mediated disorders.

In one aspect, the invention provides a method of generating IL-17 producing T-cells by first obtaining naïve T-cells from a mammal, next, exposing the T-cells to conditioned media from lipopolysaccharide (LPS)-stimulated peripheral blood mononucleocytes (PBMCs), exposing the T-cells to a T-cell receptor/CD28 T-cell stimulus in the presence of TGFβ and IL-6 and to one or more of anti IFNγ, anti IL-4, and IL-12. In one embodiment, the mammal is a primate, and in another a human.

In a second aspect, the invention provides a method of generating IL-17 producing T-cells by first obtaining peripheral blood PBMCs from a mammal, exposing the PBMCs to anti CD3, LPS, TGFβ, one or more of anti IFNγ, anti IL-4, and IL-12, anti CD28 and IL-2. In one embodiment, the mammal is a primate, and in another, a human.

In a third aspect the invention provides an isolated human IL-17 producing T-cell. In one embodiment, IL-17 producing T-cells were produced by exposing naïve human T-cells to conditioned media from LPS-stimulated PBMCs, exposing the T-cells to a T-cell receptor/CD28 T-cell stimulus, in the presence of TGFβ and IL-6 and to one or more of anti IFNγ, anti IL-4, and IL-12. In another embodiment, the IL-17 producing cells were produced by exposing mammalian PBMCs to anti CD3, lipopolysaccharide (LPS), TGFβ, one or more of anti IFNγ, anti IL-4, and IL-12, anti CD28 and IL-2.

In a fourth aspect the invention provides a method for identifying a modulator of IL-17 production in T-cells by contacting cells mammalian IL-17 producing cells with a candidate agent and measuring the amount of IL-17 produced by the cells.

In a fifth aspect, the invention provides another method for identifying a modulator of IL-17 production in T-cells by first obtaining naïve T-cells from a mammal, next, exposing the T-cells to conditioned media from LPS-stimulated PBMCs, exposing the cells to a T-cell receptor/CD28 T-cell stimulus, LPS, TGFβ and to one or more of anti IFNγ, anti IL-4, and IL-12; next, contacting the cells with a candidate agent; exposing the T-cells to IL-6; and measuring the amount of IL-17 produced by the cells.

In a sixth aspect, the invention provides yet another method for identifying a modulator of IL-17 production in T-cells by first obtaining peripheral blood mononucleocytes (PBMCs) from a mammal, next exposing the PBMCs to anti CD3, LPS, TGFβ, to one or more of anti IFNγ, anti IL-4, and IL-12; exposing the PBMCs to anti CD28; contacting the cells with a candidate agent; exposing the PBMCs to IL-2; and measuring the amount of IL-17 produced by the cells.

In a seventh aspect, the invention provides an additional method of identifying a modulation of IL-17 production in T-cells by obtaining peripheral blood mononucleocytes (PBMCs) from a mammal, purifying memory T-cells from PBMCs, exposing the memory T-cells to anti CD3 and anti CD28, contacting the cells with a candidate agent and measuring the amount of IL-17 produced by the cells. In an embodiment, the memory T-cells are isolated using negative selection.

In embodiments of the fourth fifth, sixth and seventh aspects, the methods further comprise a step of identifying the candidate agent as a modulator of IL-17 production if the amount of IL-17 is higher or lower in the presence than in the absence of the candidate molecule. In other embodiments, the amount of IL-17 is measured by ELISA. In still other embodiments the candidate agent is a small molecule, a non-peptide small organic molecule, an oligonucleotide, a peptide, a polypeptide or an antibody. In yet other embodiments, the cells are from a human.

In other aspects the invention provides modulators of IL-17 production identified by a method described in the fourth, fifth, sixth or seventh aspects.

In another aspect, the invention provides a method for modulating interleukin-17 production by T-cells by treating T-cells with a modulator of IL-17 production. The modulator can decrease or increase IL-17 production in T-cells. The modulator can be a modulator identified by a method described in the fourth, fifth, sixth or seventh aspects.

In yet other aspects, the invention provides methods of treating an IL-17-mediated disorder in a cell or a mammal by administering to the cell or mammal an effective amount of a modulator of IL-17 production in T-cells. The IL-17 mediated disorder can be an inflammatory disorder, multiple sclerosis, rheumatoid arthritis or psoriasis. The modulator can be a modulator identified by a method described in the fourth, fifth, sixth or seventh aspects.

### BRIEF DESCRIPTION OF THE DRAWINGS

FIG. 1 is a graph of results of FACS analysis of unstimulated TH-17 cells (A) or TH-17 cells stimulated with PMA.

FIG. 2 is a graph of the production of IL-17 from TH-17 cells stimulated with a T-cell receptor (TCR/CD38) stimulus and from unstimulated TH17 cells.

FIG. 3 contains graphs showing the results of FACS analyses of IL-17 secreting cells in human peripheral blood monocyte preparations.

### DETAILED DESCRIPTION

The present invention provides (1) methods for producing TH-17 cells, (2) methods for identifying modulators of IL-17 production in T-cells, and (3) methods for treating IL-17 mediated disorders. The methods include procedures for isolating and producing human TH-17 cells and using these cells to identify modulators of IL-17 production. Using human cells is advantageous for identifying modulators of IL-17 production that can be used to treat inflammatory diseases in humans.

### I. Isolation and Differentiation of TH-17 Cells

Conditions for in vitro polarization of naïve mouse T cells to a TH-17 phenotype have been described (Park, et al., Nat. Immunol, 6:1133-41, 2006; Harrington et al., Curr. Opin. Immunol., 18:349-56, 2006; Veldhoen et al. Immunity, 24:179-89, 2006, Mangan et al., Nature, 441:231-4, 2006) and are dependent on T-cell receptor (TCR)/CD28 stimulation in the presence of cytokines TGFβ and IL-6 and blockade of IL-4, IL-12 and IFNγ signaling. However, production of human TH-17 cells or TH-17 cells from other species may require different factors or conditions.

The type of TH response in vivo, is dictated by the type of cytokines and other factors secreted by cells of the immune system, particularly dendritic cells (DCs), in response to particular pathogens (Veldhoen et al. J. Immunology, 176:6202-10, 2006). For this reason, naïve T-cells are stimulated in the presence of factors secreted by endogenous DCs and other cells of the innate immune system present in human peripheral blood mononuclear cell (PBMC) preparations. Several methods have been developed, where lipopolysaccharide (LPS) is used as a pro-inflammatory stimulus for endogenous DCs and other cells of the innate immune system.

In one method, peripheral blood mononuclear cell (PBMC) preparations (which contain both naïve T cells and DCs) are stimulated in the presence of a TCR/CD28 T-cell stimulus and LPS as a pro-inflammatory DC stimulus. Exogenous IL-6 and TGFβ and blocking antibodies for IL-4, IL-12 and IFNγ are also added to ensure blockade of TH-1 and TH-2 differentiation.

In another method, purified naïve T-cells are incubated in conditioned media from LPS-stimulated PBMCs in the presence of a TCR/CD28 T cell stimulus and LPS as a pro-inflammatory DC stimulus. Exogenous IL-6, TGFβ and blocking antibodies for IL-4, IL-12 and IFNγ are also added to ensure blockade of TH-1 and TH-2 differentiation.

In yet another method, human memory T-cells are purified from human PBMCs using negative selection. Memory T-cells are CD4 positive and CD45RO positive. They can be stimulated with anti CD3 and anti CD28 to produce IL-17. This method provides cells for measuring TH-17 cell function without having to conduct time consuming and expensive in vitro polarization of naïve T-cells into a TH-17 phenotype.

An “IL-17-producing cell” includes a progenitor or precursor cell that is committed in a pathway of cell development or cell differentiation to differentiating into an IL-17-producing cell. A progenitor or precursor cell to the IL-17 producing cell can be found in a draining lymph node (DLN) or in peripheral blood. Additionally, “IL-17-producing cell” encompasses an IL-17-producing cell, that has been, e.g., activated, e.g., by lipopolysaccharide, phorbol ester, ionophore, carcinogen and/or anti-CD antibody, further differentiated, stored, frozen, dessicated, inactivated, partially degraded, e.g., by apoptosis, proteolysis, or lipid oxidation, or modified, e.g., by recombinant technology.

“Purified cell” encompasses, e.g., one or more “IL-17 producing cells” that is substantially free of other types of cells, e.g., contamination by other types of T cells, including TH-1 cells, producing integrin-γ and TH-2 cells, which produce IL-4. Purity can be assessed by use of a volume that is defined by geometric coordinates or by a compartment comprising, e.g., a flask, tube, or vial. A “purified L-17 producing cell” can be defined by, e.g., a compartment where the “IL-17 producing cells” constitute at least 20% of all the cells, at least 30% of all the cells, at least 40% of all the cells, at least 50% of all the cells, at least 60% of all the cells, at least 70% of all the cells, at least 80% of all the cells, at least 90% of all the cells; or at least 95% of all the cells.

IL-17 producing cells can be characterized by cytokine secretion, including IL-17 and by cell surface markers, e.g., CD45RB, which is lower for IL-17 producing cells than for IFNγ producing cells. Other markers include variants of IL-17, including IL-17F (aka IL-75), markers from tables 10 A and B from U.S. Patent Publication No. 2006/0140950, which is incorporated herein by reference.

Expression of markers can be measured, e.g., by measuring mRNA levels or polypeptide levels. Methods include, without limitation, quantitative RT-PCR, ELISA, immunoprecipitation followed by western blotting and mass spectrometry.

As used herein, a “mammal” refers to an animal from the kingdom animalia, the phylum chordate and the class mammalia. “Mammal” includes, without limitation, humans, domestic and farm animals, and zoo, sports or pet animals, such as sheep, goats, pigs, dogs, horses, cats, cows, etc.

### II. Methods for Identifying Modulators of IL-17 Production

The present invention includes methods to identify modulators of IL-17 production. Assays to identify molecules that modulate IL-17 production in T-cells can be amenable to high-throughput screening of chemical and other libraries. The assays provided are generally cell-based assays. Furthermore, these assays measure the effects of candidate agents on IL-17 production following secondary stimulation of T-cells that may have already undergone primary stimulation under polarising conditions.

In one embodiment, cells capable of expressing IL-17 (TH-17 cells) are contacted with a candidate agent, and the ability of the candidate agent to alter expression of the IL-17 polypeptide or nucleic acid is determined by comparison to a reference range or control. For example, TH-17 cells are contacted with a candidate agent or a control agent and the ability of the candidate agent to alter the expression of IL-17 polypeptides or nucleic acids is determined by comparing the difference in the level of expression of the IL-17 polypeptides or nucleic acids between the treated and control cells. The assays include (1) cells capable of producing IL-17, (2) candidate agents or molecules and (3) methods for measuring levels of IL-17 or other markers.

A. TH-17 Cells

TH-17 cells can be prepared from T-cells from any mammal. In one embodiment, the TH-17 cells are prepared from human PBMCs or T-cells as described above and in Example 1. In another embodiment TH-17 cells are prepared from human PBMCs as described above and in Example 3.

B. Candidate Agents

As used herein, an “agent” refers to a substance such as a chemical compound (naturally occurring or synthesized), such as a biological macromolecule, such as a nucleic acid (e.g., DNA, RNA antisense RNA, siRNA, and ribozymes), peptide, polypeptide, peptidomimetic, protein, non-peptide, antibody or fragment thereof, lipid, carbohydrate, small molecule, organic molecule, other drug or an extract made from biological materials such as bacteria, plants, fungi or animal cells or tissues, or an inorganic element or molecules. Agents include inhibitors and antagonists or activators and agonists, which refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a composition that reduces, blocks, or inactivates a constitutive activity. An “agonist” is a compound that interacts with a target to cause or promote an increase in the activation of the target. An “antagonist” is a compound that opposes the actions of an agonist. An antagonist prevents, reduces, inhibits or neutralizes the activity of an agonist. An antagonist can also prevent, inhibit or reduce constitutive activity of a target, e.g., a target receptor, even where there is no identified agonist.

A modulator of IL-17 production may partially or completely modulate IL-17 production. Modulators include, without limitation, molecules that can modulate directly or indirectly IL-17 expression, activity and/or function.

The term “agent” also includes combinations of agents, including, without limitation, known anti-inflammatory agents. For example, known treatments for multiple sclerosis may be combined with other agents, such as small organic compound libraries to be screened in the assay.

Candidate agents include a plurality (e.g. a library) of candidate agents. Candidate agents can be obtained using any of the numerous approaches in combinatorial library methods known in the art, including biological libraries, spatially addressable parallel solid phase or solution phase libraries, synthetic library methods requiring deconvolution, the “one-bead one-compound” library method and synthetic library methods using affinity chromatography selection. The biological library approach is suited to peptide libraries, while the other four approaches are applicable to peptide, non-peptide, oligomer or small molecule libraries of compounds (Lam, Anticancer Drug Des., 12:145, 1997; U.S. Pat. Nos. 5,738,683 and 5,807,683).

1. Small Molecules

The term “small molecule” refers to compounds that are not macromolecules (see, e.g., Karp, Bioinformatics Ontology 16:269-85, 2000; Verkman. Am J Physiol-Cell Physiol., 286:465-74, 2004). Thus, small molecules are often considered those compounds that are, e.g., less than one thousand Daltons (e.g., Voet and Voet, Biochemistry, 2nd ed., ed. N. Rose, Wiley and Sons, New York, 14, 1995). For example, Davis et al. (Proc. Natl. Acad. Sci. USA 102:5981-86, 2005) use the phrase “small molecule” to indicate folates, methotrexate and neuropeptides, while Halpin and Harbury (PLos Biology 2:1022-30, 2004) use the phrase to indicate small molecule gene products, e.g., DNAs, RNAs and peptides. Examples of natural small molecules include, without limitation, cholesterols, neurotransmitters and siRNAs. Synthesized small molecules include, without limitation, various chemicals listed in numerous commercially available small molecule databases, e.g., FCD (Fine Chemicals Database), SMID (Small Molecule Interaction Database), ChEBI (Chemical Entities of Biological Interest), and CSD (Cambridge Structural Database). (See, e.g., Alfarano et al. Nucl. Acids Res. Database Issue 33:D416-24, 2005.)

Small molecules include, without limitation, inorganic molecules, organic molecules, organic molecules containing an inorganic component, molecules comprising a radioactive atom(s), synthetic molecules, peptide mimetics and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins have been described. (See, e.g., Casset, et al., Biochem. Biophys. Res. Commun. 307:198-205, 2003; Muyldermans, J. Biotechnol. 74:277-302, 2001; Li, Nat. Biotechnol. 18:1251-1256, 2000; Apostolopoulos, et al., Curr. Med. Chem. 9:411-420, 2002; Monfardini, et al., Curr. Pharm. Des. 8:2185-2199, 2002; Domingues, et al., Nat. Struct. Biol. 6:652-656, 1999; Sato and Sone, Biochem. J. 371:603-608, 2003; U.S. Pat. No. 6,326,482 issued to Stewart, et al.)

Small molecules include organic molecules that can be part of combinatorial libraries. There are a number of methods of producing combinatorial libraries. Examples of suitable methods based on the present description for the synthesis of molecular libraries can be found in the art, for example in DeWitt, et al., Proc. Natl. Acad. Sci. USA, 90:6906, 1993; Erb, et al., Proc. Natl. Acad. Sci. USA, 91:11422, 1994; Zuckermann, et al., J. Med. Chem., 37:2768, 1994; Cho, et al., Science, 261:1303, 1993; Carrell et al., Agnew. Chem. Int. Ed. Engl. 33:2059, 1994; Carrell et al., Agnew. Chem. Int. Ed. Engl., 33:2061, 1994; and Gallop, et al., J. Med. Chem. 37:1233, 1994.

2. Nucleic Acids

Various nucleic acid molecules can also be candidate agents for modulation of IL-17 expression in TH-17 cells. Altered expression of IL-17 genes or other genes in TH-17 cells may be achieved in a cell or organism through the use of various inhibitory polynucleotides, such as antisense polynucleotides, siRNAs, and ribozymes that bind and/or cleave the mRNA transcribed from genes involved in IL-17 production. (See, e.g., Galderisi et al., J. Cell Physiol. 181:251-57, 1999; Sioud, Curr. Mol. Med. 1:575-88, 2001.) Inhibitory polynucleotides to, e.g., IL-17 , may also be useful as IL-17 signaling antagonists and, as such, may also be useful in preventing or treating disorders related to IL-17 signaling.

Inhibitory polynucleotides may also consist of aptamers, i.e., polynucleotides that bind to and regulate protein activity, e.g., the activity of IL-17. Aptamers are described throughout the literature. (See, e.g., Nimjee et al., Annu. Rev Med. 56:555-83, 2005 and Patel, Curr. Opin. Chem. Biol. 1:32-46, 1997.)

Antisense polynucleotides or ribozymes may be complementary to an entire coding strand of a gene, or to only a portion thereof. Alternatively, antisense polynucleotides or ribozymes can be complementary to a non-coding region of the coding strand of a gene. Antisense polynucleotides or ribozymes can be constructed using chemical synthesis and enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, without limitation, phosphorothioate, methylphosphonate, phosphoroamidate, boranophosphate, morpholino, and peptide nucleic acid (PNA) linkages (Galderisi et al., supra; Heasman, Dev. Biol. 243:209-14, 2002; Micklefield, Curr. Med. Chem. 8:1157-79, 2001). Alternatively, these molecules can be produced biologically using an expression vector into which a polynucleotide related to the present invention has been subcloned in an antisense (i.e., reverse) orientation.

The nucleic acid candidate agents can also include triplex-forming oligonucleotides (TFOs) that bind in the major groove of duplex DNA with high specificity and affinity (Knauert and Glazer, Hum. Mol. Genet. 10:2243-51, 2001). Expression of genes in TH-17 cells can be inhibited by targeting TFOs complementary to the regulatory regions of the genes (i.e., the promoter and/or enhancer sequences) to form triple helical structures that prevent transcription of the genes.

In addition, double stranded DNA molecules can also be candidate agents. These include decoy DNA molecules that may interact with molecules that regulate gene expression in TH-17 cells.

Nucleic acids candidate agents also include short interfering RNA (siRNA) molecules. siRNA molecules are short, e.g., 19-25 nucleotides, double-stranded RNA molecules that cause sequence-specific degradation of target mRNA. This degradation is known as RNA interference (RNAi). (See, e.g., Bass, Nature 411:428-29, 2001.) Originally identified in lower organisms, RNAi has been effectively applied to mammalian cells and has been shown to prevent fulminant hepatitis in mice treated with siRNA molecules targeted to Fas mRNA (Song et al., Nature Med. 9:347-51, 2001). In addition, intrathecally delivered siRNA has been reported to block pain responses in two models (agonist-induced pain model and neuropathic pain model) in the rat (Dom et al., Nucleic Acids Res. 32:e49, 2004).

siRNA molecules maybe generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al., Proc. Natl. Acad. Sci. USA 99:6047-52, 2002). siRNA molecules may be chemically synthesized (Elbashir et al., Nature 411:494-98, 2001) or produced by in vitro transcription using single-stranded DNA templates (Yu et al., supra). Alternatively, siRNA molecules can be produced biologically, either transiently (Yu et al., supra; Sui et al., Proc. Natl. Acad. Sci. USA 99:55 15-20, 2002) or stably (Paddison et al., Proc. Natl. Acad. Sci. USA 99:1443-48, 2002), using an expression vector(s) containing sense and antisense siRNA sequences. Reduction of levels of target mRNA in primary human cells, in an efficient and sequence-specific manner, has been demonstrated using adenoviral vectors that express hairpin RNAs, which are further processed into siRNAs (Arts et al., Genome Res. 13:2325-32, 2003).

siRNA molecules can be designed based on criteria well known in the art. (See, e.g., Elbashir et al., EMBO J. 20:6877-88, 2001). For example, the target segment of the target mRNA may begin with AA, TA, GA, or CA; the GC ratio of the siRNA molecule can be 45-55%; the siRNA molecule may not contain three of the same nucleotides in a row; the siRNA molecule may not contain seven mixed G/Cs in a row; and the target segment may be in the ORF region of the target mRNA and may be at least 75 bp after the initiation ATG and at least 75 bp before the stop codon. Based on these criteria, or on other known criteria (e.g., Reynolds et al., Nature Biotechnol 22:326-30, 2004), siRNA molecules that target specific mRNA polynucleotides may be designed by a skilled artisan.

In one example, the candidate nucleic acid agent may target IL-17 mRNA. IL 17 nucleic acids may be obtained using standard cloning techniques from, for example, genomic DNA or cDNA or can be synthesized using well known and commercially available techniques. The nucleic acids may contain one or more nucleotide substitutions, additions or deletions into the nucleotide sequence. Standard techniques known to skilled artisans can be used to introduce mutations, including, for example, site-directed mutagenesis and PCR-mediated mutagenesis.

3. Antibodies

Candidate agents can also be antibodies, or fragments thereof. The antibodies may be polyclonal, monoclonal,

human, humanized, chimeric, or in vitro-generated antibodies. As used herein, the term “antibody” refers to a protein comprising at least one or two, heavy (H) chain variable regions (abbreviated herein as VH), and at least one or two light (L) chain variable regions (abbreviated herein as VL). The VH and VL regions can be further subdivided into regions of hypervariability, termed “complementarity determining regions” (“CDRs”), interspersed with regions that are more conserved, termed “framework regions” (“FR”). The extent of the FRs and CDRs has been precisely defined. (See, Kabat et al., Sequences of Proteins of Immunological Interest, Fifth Edition, U.S. Department of Health and Human Services, NIH Publication No. 91-3242, 1991; and Chothia et al., J. Mol. Biol. 196:901-917, 1987, which are incorporated herein by reference.) Each VH and VL is composed of three CDRs and four FRs, arranged from amino-terminus to carboxy-terminus in the following order: FR1, CDR1, FR2, CDR2, FR3, CDR3, FR4. The antibody may further include a heavy and light chain constant region to thereby form a heavy and light immunoglobulin chain, respectively. In one embodiment, the antibody is a tetramer of two heavy immunoglobulin chains and two light immunoglobulin chains, wherein the heavy and light immunoglobulin chains are interconnected, e.g., by disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Cl ci′) of the classical complement system.

“Immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 kDa, or 214 amino acids) are encoded by a variable region gene at the NH2-terminus (about 110 amino acids) and a kappa or lambda constant region gene at the COOH-terminus. Full-length immunoglobulin “heavy chains” (about 50 kDa, or 446 amino acids), are similarly encoded by a variable region gene (about 116 amino acids) and one of the other aforementioned constant region genes, e.g., gamma (encoding about 330 amino acids). The immunoglobulin heavy chain constant region genes encode for the antibody class, i.e., isotype (e.g., IgM or IgG). The antigen binding fragment of an antibody (or simply “antibody portion,” or “fragment”), as used herein, refers to one or more fragments of a full-length antibody that retain the ability to specifically bind to an antigen (e.g., CD3). Examples of binding fragments encompassed within the term “antigen binding fragment” of an antibody include, without limitation, (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VII domain; and (vi) an isolated complementarity determining region (CDR). Furthermore, although the two domains of the Fv fragment, VL and VII, are encoded by separate genes, they may be joined, using recombinant methods, by a synthetic linker that enables them to be made as a single protein chain in which the VL and VII regions pair to form monovalent molecules (known as single chain Fv (scFv)). Such single chain antibodies are also intended to be encompassed within the term “antigen binding fragment” of an antibody. These antibody fragments are obtained using conventional techniques known to those skilled in the art, and the fragments are screened for utility in the same manner as are intact antibodies.

Antibody molecules may be produced by methods well known to those skilled in the art. For example, monoclonal antibodies maybe produced by generation of hybridomas in accordance with known methods. Hybridomas formed in this manner can be screened using an assay of the present invention. For example, lysed cells, cell extracts, subcellular fractions (including membranes, mitochondria, etc.), protein fractions, or cell culture supernatants from IL-17 producing cells may be used to immunize animals to obtain polyclonal and monoclonal antibodies that interact with molecules of IL-17 producing cells and which may inhibit IL-17 production by interacting with specific molecules from IL-17 producing cells.

Monoclonal antibodies may be generated by other methods known to those skilled in the art of recombinant DNA technology. As an alternative to preparing monoclonal antibody-secreting hybridomas, a monoclonal antibody to a molecule from IL-17 producing cells may be identified and isolated by screening a recombinant combinatorial immunoglobulin library (e.g., an antibody phage display library) using a screening assay of the present invention to thereby isolate immunoglobulin library members that bind to molecules that modulate IL-17 production. Techniques and commercially available kits for generating and screening phage display libraries are well known to those skilled in the art. Additionally, examples of methods and reagents particularly amenable for use in generating and screening antibody display libraries can be found in the literature. For example, the “combinatorial antibody display” method is well known and was developed to identify and isolate antibody fragments having particular antigen specificities, and can be used to produce monoclonal antibodies. After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primers to a conserved 3′ constant region, can be used for PCR amplification of the heavy and light chain variable regions from a number of murine antibodies; a similar strategy has also been used to amplify human heavy and light chain variable regions from human antibodies.

Polyclonal sera and antibodies may be produced by immunizing a suitable subject with IL-17 producing cells or extracts thereof. The antibody titer in the immunized subject may be monitored over time by standard techniques, such as with ELISA using immobilized protein. If desired, the antibody molecules directed against a molecule from IL-17 producing cells may be isolated from the subject or culture media and further purified by well-known techniques, such as protein A chromatography, to obtain an IgG fraction.

Fragments of antibodies to polypeptides may be produced by cleavage of the antibodies in accordance with methods well known in the art. For example, immunologically active Fab and F(ab′)2 fragments may be generated by treating the antibodies with an enzyme such as pepsin.

Additionally, chimeric, humanized, and single-chain antibodies to polypeptides, comprising both human and nonhuman portions, may be produced using standard recombinant DNA techniques and/or a recombinant combinatorial immunoglobulin library. Humanized antibodies may also be produced using transgenic mice which are incapable of expressing endogenous immunoglobulin heavy and light chain genes, but which can express human heavy and light chain genes. For example, human monoclonal antibodies (mAbs) directed against, molecules from IL-17 producing cells, may be generated using transgenic mice carrying the human immunoglobulin genes rather than murine immunoglobulin genes. Splenocytes from these transgenic mice immunized with the antigen of interest may then be used to produce hybridomas that secrete human mAbs with specific affinities for epitopes from a human protein.

Chimeric antibodies, including chimeric immunoglobulin chains, maybe produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted.

An antibody or an immunoglobulin chain maybe humanized by methods known in the art. Humanized antibodies, including humanized immunoglobulin chains, may be generated by replacing sequences of the Fv variable region that are not directly involved in antigen binding with equivalent sequences from human Fv variable regions. General methods for generating humanized antibodies are provided by Morrison (Science 229:1202-07, 1985), Oi et al. (BioTechniques 4:214, 1986), Queen et al. (U.S. Pat. Nos. 5,585,089; 5,693,761; 5,693,762), the contents of all of which are incorporated herein by reference. The methods include isolating, manipulating, and expressing the nucleic acid sequences that encode all or part of immunoglobulin Fv variable regions from at least one of a heavy or light chain. Sources of such nucleic acid sequences are well known to skilled artisans and, for example, may be obtained from a hybridoma producing an antibody against a predetermined target. The recombinant DNA encoding the humanized antibody, or fragment thereof, then can be cloned into an appropriate expression vector.

Humanized or CDR-grafted antibody molecules or immunoglobulins may be produced by CDR grafting or CDR substitution, wherein one, two, or all CDRs of an immunoglobulin chain can be replaced. (See, e.g., U.S. Pat. No. 5,225,539; Jones et al., Nature 321:552-25, 1986; Verhoeyan et al., Science 239:1534, 1988; Beidler et al., J. Immunol. 141:4053-60, 1988; Winter, U.S. Pat. No. 5,225,539, the contents of all of which are incorporated herein by reference.) Winter describes a CDR-grafting method that can be used to prepare humanized antibodies (UK Patent Application GB 2188638A; Winter, U.S. Pat. No. 5,225,539, the contents of which are incorporated herein by reference). All CDRs of a particular human antibody may be replaced with at least a portion of a nonhuman CDR, or only some CDRs maybe replaced with nonhuman CDRs. It is necessary to replace only the number of CDRs required for binding of the humanized antibody to a predetermined antigen.

Human antibodies may additionally be produced using transgenic nonhuman animals that are modified to produce fully human antibodies rather than the animal's endogenous antibodies in response to challenge by an antigen. (See, e.g., PCT publication WO 94/02602.) The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal that provides all the desired modifications is then obtained as progeny by crossbreeding intermediate transgenic animals containing fewer than the full complement of the modifications. An embodiment of such a nonhuman animal is a mouse, and is termed the XENOMOUSE as disclosed in PCT publications WO 96/33735 and WO 96/34096. This animal produces B cells that secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

Monoclonal, chimeric and humanized antibodies that have been modified by, e.g., deleting, adding, or substituting other portions of the antibody, e.g., the constant region, are also within the scope of candidate agents. As nonlimiting examples, an antibody can be modified by deleting the constant region, by replacing the constant region with another constant region, e.g., a constant region meant to increase half-life, stability, or affinity of the antibody, or a constant region from another species or antibody class, and by modifying one or more amino acids in the constant region to alter, for example, the number of glycosylation sites, effector cell function, Fc receptor (FcR) binding, complement fixation, etc.

Methods for altering an antibody constant region are known in the art. Antibodies with altered function, e.g., altered affinity for an effector ligand, such as FcR on a cell, or the C-1 component of complement, can be produced by replacing at least one amino acid residue in the constant portion of the antibody with a different residue. (See, e.g., European Patent Publication EP 388,151 A1 and U.S. Pat. Nos. 5,624,821 and 5,648,260, the contents of all of which are incorporated herein by reference.) Similar types of alterations to murine (or other species') immunoglobulins may he applied to reduce or eliminate these functions, and are known in the art.

For example, it is possible to alter the affinity of an Fc region of an antibody (e.g., an IgG, such as a human IgG) for an FcR (e.g., Fc gamma Ri), or for C-lq binding by replacing the specified residue(s) with a residue(s) having an appropriate functionality on its side chain, or by introducing a charged functional group, such as glutamate or aspartate, or an aromatic nonpolar residue such as phenylalanine, tyrosine, tryptophan or alanine. (See, e.g., U.S. Pat. No. 5,624,821.)

Antibody-based molecules may also be candidate agents. Such antibody-based molecules include small modular immunopharmaceutical (SMIP) drugs (Trubion Pharmaceuticals, Seattle, Wash.). SMIPs are single-chain polypeptides composed of a binding domain for a cognate structure such as an antigen, a counterreceptor or the like, a hinge-region polypeptide having either one or no cysteine residues, and immunoglobulin CR2 and CR3 domains. (See also www.trubion.com.) SMIPs exhibit the binding specificity and activity of monoclonal antibodies, but are approximately one-third to one-half the size of conventional therapeutic monoclonal antibodies, and have an extensive in vivo half-life. SMIPs and their uses and applications are disclosed in, e.g., U.S. Patent Publication. Nos. 2003/0118592, 2003/0133939, 2004/005 8445, 2005/0136049, 2005/0175614, 2005/0180970, 2005/0186216, 2005/0202012, 2005/0202023, 2005/0202028, 2005/0202534, and 2005/023 8646, and related patent family members thereof, all of which are incorporated herein by reference in their entireties.

C. Delivery of Candidate Agents to Cells

Libraries of compounds may be presented, for example, in solution (e.g., Houghten, Bio/Techniques, 13:412-421, 1992), or on beads (Lam, Nature, 354:82-84, 1991), chips (Fodor, Nature 364:555-56, 1993), bacteria (U.S. Pat. No. 5,223,409), spores (U.S. Pat. Nos. 5,571,698; 5,403,484; and 5,223,409), plasmids (Cull et al., Proc. Natl. Acad. Sci. USA 89:1865-69, 1992) or phage (Scott and Smith, Science 249:386-390, 1990; Devlin, Science; 249:404-406, 1990; Cwirla et al., Proc. Natl. Acad. Sci. USA 87:6378-82, 1990; and Felici, J. Mol. Biol. 222:301-10, 1991).

Nucleic acids may be delivered to cells using a number of delivery methods, including, without limitation, liposomes, dendrimers, or in vectors, such as, without limitation, plasmids, or adeno, lenti or other viruses.

In one embodiment, the assays are designed to measure the effects of candidate agents on IL-17 production following secondary stimulation of T-cells that have undergone primary stimulation under polarising conditions. To achieve this, candidate agents can be added to T-cells after primary stimulation under polarising conditions, but before or at the time of secondary stimulation of T-cells. Primary stimulation can include, without limitation, treating cells with a TCR/CD28 stimulus and/or LPS. Secondary stimulation can include, without limitation, stimulation with IL-2, IL-6 and/or IL-23.

In another embodiment, the assays measure the effects of candidate agents on IL-17 production in memory T-cells during or following stimulation of the cells. Stimulation can include, without limitation, treatment with anti CD3 and anti CD28.

D. Measurement of the Effect of Candidate Agents

The ability of the candidate agents to alter the expression of IL-17 polypeptide or nucleic acid can be determined by methods known to those of skill in the art, for example and without limitation, by flow cytometry, ELISA, radiolabelling, a scintillation assay, immunoprecipitation, western blot analysis, northern blot analysis or RT-PCR.

For example, mRNA can be directly detected and quantified using hybridization-based assays, such as northern hybridization, in situ hybridization, dot and slot blots, and oligonucleotide arrays. Hybridization-based assays refer to assays in which a probe nucleic acid is hybridized to a target nucleic acid. In some formats, the target, the probe, or both are immobilized. The immobilized nucleic acid may be DNA, RNA, or another oligonucleotide or polynucleotide, and may comprise naturally or non-naturally occurring nucleotides, nucleotide analogs, or oligonucleotides containing non-naturally occurring backbones. Methods of selecting nucleic acid probe sequences to detect IL-17 message are based on the nucleic acid sequence of IL-17, and are well known in the art. (See GenBank accession numbers NM 002190, NM 052872.)

Alternatively, mRNA can be amplified before detection and quantitation. Such amplification-based assays are well known in the art and include polymerase chain reaction (PCR), reverse-transcription-PCR (RT-PCR), PCR-enzyme-linked immunosorbent assay (PCR-ELISA), and ligase chain reaction (LCR). Primers and probes for producing and detecting amplified IL-17 gene products (e.g., mRNA or cDNA) maybe readily designed and produced without undue experimentation by those of skill in the art based on the nucleic acid sequences of IL-17 and its variants. As a nonlimiting example, amplified IL-17 gene products maybe directly analyzed, for example, by gel electrophoresis; by hybridization to a probe nucleic acid; by sequencing; by detection of a fluorescent, phosphorescent, or radioactive signal; or by any of a variety of well-known methods. In addition, methods are known to those of skill in the art for increasing the signal produced by amplification of target nucleic acid sequences. A skilled artisan will recognize that whichever amplification method is used, a variety of quantitative methods known in the art (e.g., quantitative PCR) may be used if quantitation of gene products is desired.

IL-17 polypeptides (or fragments thereof) may be detected using various well-known immunological assays employing anti-IL-17 antibodies that may be generated or are commercially available. Immunological assays refer to assays that utilize an antibody (e.g., polyclonal, monoclonal, chimeric, humanized, scFv, and/or fragments thereof) that specifically binds to, e. g., an IL-17 polypeptide (or a fragment thereof). Such well-known immunological assays suitable for the practice of the present invention include ELISA, radioimmunoassay (RIA), immunoprecipitation, immunofluorescence, fluorescence-activated cell sorting (FACS), and western blotting. An IL-17 polypeptide may also be detected using a labeled IL-17 receptor polypeptide.

Candidate agents can also be screened using an assay in which regulatory regions of the IL-17 gene or regulatory regions of other genes expressed in TH-17 cells are fused to a reporter gene, such as a luciferase, a fluorescent protein, including green fluorescent protein, a reporter detectable with magnetic resonance imaging, a reporter detectable by PET or SPECT, or a reporter detectable with visible light.

To examine the extent of inhibition, for example, assays comprising TH-17 cells are treated with a candidate agent and are compared to control samples without the agent or with a control agent. Control samples, are assigned a relative activity value of 100%. Inhibition is achieved when the activity value relative to the control is about 90% or less, 85% or less, 80% or less, 75% or less, 70% or less, 65% or less, 60% or less, 55% or less, 50% or less, 45% or less, 40% or less, 35% or less, 30% or less, 25% or less, or less than 25%. Activation is achieved when the activity value relative to the control is about 110%, at least 120%, at least 140%, at least 160%, at least 180%, at least 2-fold, at least 2.5-fold, at least 5-fold, at least 10-fold, at least 20-fold, at least 40-fold, or over 40-fold higher.

### III. Treatment of IL-17 Mediated Disorders

Modulators of IL-17, IL-17 production or IL-17 signaling can be used to treat and diagnose a number of disorders and conditions, e.g., of the central nervous system, peripheral nervous system, and gastrointestinal tract. Antagonists of IL-17 production can be used to treat disorders mediated by IL-17, including inflammatory conditions, characterized by elevated levels of IL-17. Molecules that increase IL-17 production may be used to stimulate a protective immune response to infections, such as infections by Mycobacterium tuberculosis.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also mean in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an IL-17 modulator with a human or animal subject, a cell, tissue, physiological compartment, or physiological fluid. “Treatment of a cell” also encompasses situations where the IL-17 modulator contacts an IL-17 receptor, e.g., in the fluid phase or colloidal phase, but also situations where the agonist or antagonist does not contact the cell or the receptor.

A “subject” is a vertebrate. In one embodiment, a subject is a mammal and in another embodiment, a subject is a human.

“Effective amount” encompasses an amount sufficient to show a meaningful patient benefit, e.g., amelioration of symptoms of, healing of, increase in healing of, or prevention of a symptom or sign of the medical condition. Effective amount also means an amount sufficient to allow or facilitate diagnosis. An effective amount for a particular patient or veterinary subject may vary depending on factors such as the condition being treated, the overall health of the patient, the method route and dose of administration and the severity of side effects. (See, e.g., U.S. Pat. No. 5,888,530 issued to Netti, et al.) An effective amount can be the maximal dose or dosing protocol that avoids significant side effects or toxic effects. The effect will result in an improvement of a diagnostic measure or parameter by at least 5%, at least 10%, at least 20%, at least 30%, at least 40%, at least 50%, at least 60%, at least 70%, at least 80%, or at least 90%, where 100% is defined as the diagnostic parameter shown by a normal subject. (See, e.g., Maynard, et al., A Handbook of SOPs for Good Clinical Practice, Interpharm Press, Boca Raton, Fla., 1995; Dent, Good Laboratory and Good Clinical Practice, Urch Publ., London, UK, 2001.)

A. IL-17 Disorders

As used herein, the term “inflammatory disease” or “inflammatory disorder” refers to pathological states resulting in inflammation, typically caused by leukocyte infiltration. Examples of such disorders include inflammatory skin diseases, including, without limitation, psoriasis and atopic dermatitis; systemic scleroderma and sclerosis; responses associated with inflammatory bowel disease (IBD) (such as Crohn's disease and ulcerative colitis); ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms; cerebral edema secondary to stroke; cranial trauma, hypovolemic shock; asphyxia; adult respiratory distress syndrome; acute-lung injury; Behcet's Disease; dermatomyositis; polymyositis; multiple sclerosis (MS); dermatitis; meningitis; encephalitis; uveitis; osteoarthritis; lupus nephritis; autoimmune diseases such as rheumatoid arthritis (RA), Sjorgen's syndrome, vasculitis; diseases involving leukocyte diapedesis; central nervous system (CNS) inflammatory disorder, multiple organ injury syndrome secondary to septicaemia or trauma; alcoholic hepatitis; bacterial pneumonia; antigen-antibody complex mediated diseases including glomerulonephritis; sepsis; sarcoidosis; immunopathologic responses to tissue or organ transplantation; inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis; etc. Indications include, without limitation, chronic inflammation, autoimmune diabetes, rheumatoid arthritis (RA), rheumatoid spondylitis, gouty arthritis and other arthritic conditions, multiple sclerosis (MS), asthma, systemic lupus erythrematosis, adult respiratory distress syndrome, Behcet's disease, psoriasis, chronic pulmonary inflammatory disease, graft versus host reaction, Crohn's Disease, ulcerative colitis, inflammatory bowel disease (IBD), which includes celiac disease and irritable bowel syndrome; Alzheimer's disease, and pyresis, along with any disease or disorder that relates to inflammation and related disorders.

B. Combination Therapies

IL-17 modulators may be administered in combination with other agents, including anti-inflammatory agents and other active compounds currently in use for the treatment of the target diseases and conditions. Such compounds include corticosteroids; non-steroidal anti-inflammatory drugs (NSAIDs), such as aspirin, ibuprofen, and COX-2 inhibitors, e.g. Celebrex™ and Vioxx™; disease-modifying anti-rheumatic drugs (DMARDs), such as methotrexate, leflunomide, sulfasalazine, azathiopine, cyclosporine, hydroxychloroquine, and D-penicillamine; and biological response modifiers (BRMs), such as TNF and IL-1 inhibitors. IL-17 modulators can also be administered with therapies used to treat MS, including β-interferons: interferon β-1b (Betaseron, Berlex), interferon β-1a (Avonex, Biogen; Rebif Serono) and glatimer acetate (Copasone, Teva).

IL-17 modulators may also be administered in combination with cytokines, lymphokines, or other hematopoietic factors such as M-CSF, GM-CSF, IL-1, IL-2, IL-3, IL-4, IL-5, IL-6, IL-7, IL-8, IL-9, IL-10, IL-11, IL-12, IL-14, IL-15, G-CSF, stem cell factor, and erythropoietin. Anticytokine antibodies may also be administered with the modulators. Thrombolytic or antithrombotic factors such as plasminogen activator and Factor VIII can also be co-therapies. Other anti-inflammatory agents may also be co-administered. Such additional factors and/or agents may be included in the pharmaceutical composition or administered separately to produce a synergistic effect with IL-17 modulators, or to minimize side effects caused by the IL-17 modulators. Conversely IL-17 modulators maybe included in formulations of the particular cytokine, lymphokine, other hematopoietic factor, thrombolytic or antithrombotic factor, or anti-inflammatory agent to minimize side effects of the cytokine, lymphokine, other hematopoietic factor, thrombolytic or antithrombotic factor, or anti-inflammatory agent.

IL-17 modulators can also be combined with inhibitors of, e.g. , antibodies to, cell surface molecules such as CD2, CD3, CD4, CD8, CD20 (e.g., the CD20 inhibitor rituximab (RITUXAN®)), CD25, CD28, CD30, CD40, CD45, CD69, CD80 (B7.1), CD86 (B7.2), CD90, or their ligands, including CD154 (gp39 or CD40L), or LFA-1IICAM-1 and VLA-4IVCAM-1 (Yusuf-Makagiansar et al., Med. Res. Rev. 22:146-67, 2002).

Examples of agents for co-therapy include IL-12 antagonists, such as chimeric, humanized, human or in vitro-generated antibodies (or antigen binding fragments thereof) that bind to IL-12/IL-23 p40 (including human IL-12/IL-23), e. g., the antibody disclosed in PCT publication WO 00/56772; IL-12 receptor inhibitors, e.g. , antibodies to human IL-12 receptor; and soluble fragments of the IL-12 receptor, e.g., human IL-12 receptor. Examples of IL-15 antagonists include antibodies (or antigen binding fragments thereof) against IL-15 or its receptor, e.g., chimeric, humanized, human or in vitro-generated antibodies to human IL-15 or its receptor, soluble fragments of the IL-15 receptor, and IL-15-binding proteins. Examples of IL-18 antagonists include antibodies, e.g., chimeric, humanized, human or in vitro-generated antibodies (or antigen binding fragments thereof), to human IL-18, soluble fragments of the IL-18 receptor, and IL-18 binding proteins (IL-18BP). Examples of IL-1 antagonists include interleukin-1-converting enzyme (ICE) inhibitors, such as VX-740, IL-1 antagonists, e.g., IL-IRA (anikinra, KINERET, Amgen), sIL1RII (Immunex), and anti-IL-1 receptor antibodies (or antigen binding fragments thereof).

Examples of TNF antagonists include chimeric, humanized, human or in vitro-generated antibodies (or antigen binding fragments thereof) to TNF (e.g., human TNFα), such as (HUMIRA™, D2E7, human TNFα antibody), CDP-571/CDP-870/BAY-10-3356 (humanized anti-TNFα antibody; Celltech/Pharmacia), cA2 (chimeric anti-TNFα antibody; REMICADE®, Centocor); anti-TNF antibody fragments (e.g., CPD870); soluble fragments of the TNF receptors, e.g., p55 or p75 human TNF receptors or derivatives thereof, e.g., kdTNFR-IgG (75 kD TNF receptor-IgG fusion protein, ENBREL; Huminex), p55 kdTNFR-IgG (55 kD TNF receptor-IgG fusion protein (LENERCEPT®)); enzyme antagonists, e.g., TNFα converting enzyme (TACE) inhibitors (e.g., α-sulfonyl hydroxamic acid derivatives, and N-hydroxyformamide TACE inhibitors GW 3333, −005, or −022); and TNF-bp/s-TNFR (soluble TNF binding protein). Some TNF antagonists are soluble fragments of the TNF receptors, e.g., p55 or p75 human TNF receptors or derivatives thereof, e.g., 75 kdTNFR-IgG, and TNFα converting enzyme (TACE) inhibitors.

In other embodiments, IL-17 modulators may be administered in combination with one or more of the following: IL-13 antagonists, e.g., soluble IL-13 receptors (sIL-13) and/or antibodies against IL-13; IL-2 antagonists, e.g., DAB 486-IL-2 and/or DAB 389-IL-2 (IL-2 fusion proteins, Seragen), and/or antibodies to IL-2R, e.g., anti-Tac (humanized anti-IL-2R, Protein Design Labs). Another combination includes IL-17 modulators in combination with nondepleting anti-CD4 inhibitors (IDEC-CE9.1/SB 210396; nondepleting primatized anti-CD4 antibody; IDEC/SmithKline). Yet other combinations include antagonists of the costimulatory pathway CD8O (B7.1) or CD86 (B7.2), including antibodies, soluble receptors or antagonistic ligands; as well as p-selectin glycoprotein ligand (PSGL), anti-inflammatory cytokines, e. g., IL-4 (DNAX/Schering); IL-6 (SCH 52000; recombinant IL-10 DNAX/Schering); IL-13 and TGF-f3, and agonists thereof (e.g., agonist antibodies).

In other embodiments, one or more IL-17 modulators can be co-formulated with, and/or co-administered with, one or more anti-inflammatory drugs, immunosuppressants, or metabolic or enzymatic inhibitors. Nonlimiting examples of drugs or inhibitors that can be used in combination with IL-17 modulators include one or more of: nonsteroidal anti-inflammatory drug(s) (NSAIDs), e.g., ibuprofen, tenidap, naproxen, meloxicam, piroxicam, diclofenac, and indomethacin; sulfasalazine; corticosteroids such as prednisolone; cytokine suppressive anti-inflammatory drug(s) (CSAIDs); inhibitors of nucleotide biosynthesis, e.g., inhibitors of purine biosynthesis, folate antagonists (e.g., methotrexate (N-[4-[[2,4-diamino-6-pteridinyl)methyl]methylamino]benzoyl]-L-glutamic acid); and inhibitors of pyrimidine biosynthesis, e.g., dihydroorotate dehydrogenase (DHODH) inhibitors (e.g., leflunomide).

Examples of additional inhibitors include one or more of: corticosteroids (oral, inhaled and local injection); immunosuppresants, e. g., cyclosporin, tacrolimus (FK-506); and mTOR inhibitors, e.g., sirolimus (rapamycin-RAPAMUNE™ or rapamycin derivatives, e.g., soluble rapamycin derivatives (e.g., ester rapamycin derivatives, e.g., CCI-779); agents that interfere with signaling by proinflammatory cytokines such as TNFα or IL-1 (e.g. IRAK, NIK, IKK, p38 or MAP kinase inhibitors); COX2 inhibitors, e.g., celecoxib, rofecoxib, and variants thereof, phosphodiesterase inhibitors, e.g., R973401 (phosphodiesterase Type P1 inhibitor); phospholipase inhibitors, e.g., inhibitors of cytosolic phospholipase 2 (cPLA2) (e.g., trifluoromethyl ketone analogs); inhibitors of vascular endothelial cell growth factor or growth factor receptor, e.g., VEGF inhibitor and/or VEGF-R inhibitor; and inhibitors of angiogenesis.

Additional examples of therapeutic agents that can be combined with an IL-17 modulator include one or more of: 6-mercaptopurines (6-MP); azathiopine sulphasalazine; mesalazine; olsalazine; chloroquine/hydroxychioroquine (PLAQUENIL®); pencillamine; aurothiomalate (intramuscular and oral); azathioprine; coichicine; beta-2 adrenoreceptor agonists (salbutamol, terbutaline, salmeteral); xanthines (theophylline, aminophylline); cromoglycate; nedocromil; ketotifen; ipratropium and oxitropium; mycophenolate mofetil; adenosine agonists; antithrombotic agents; complement inhibitors; and adrenergic agents.

Use of IL-17 modulators in combination with other therapeutic agents to treat or prevent specific disorders is discussed in further detail below.

Nonlimiting examples of agents for treating or preventing arthritic disorders (e.g., rheumatoid arthritis, inflammatory arthritis, rheumatoid arthritis, juvenile rheumatoid arthritis, osteoarthritis and psoriatic arthritis), with which IL-17 modulators may be combined include one or more of the following: IL-12 antagonists as described herein; NSAIDs; CSAIDs; TNFs, e.g., TNFα, antagonists as described herein; nondepleting anti-CD4 antibodies as described herein; IL-2 antagonists as described herein; anti-inflammatory cytokines, e.g., IL-4, IL-6, IL-13 and TGFα, or agonists thereof; IL-1 or IL-1 receptor antagonists as described herein; phosphodiesterase inhibitors as described herein; Cox-2 inhibitors as described herein; iboprost: methotrexate; thalidomide and thalidomide-related drugs (e.g., Celgen); leflunomide; inhibitor of plasminogen activation, e.g., tranexamic acid; cytokine inhibitor, e.g., T-614; prostaglandin E1; azathiopine; inhibitors of interleukin-1 converting enzyme (ICE); zap-70 and/or Ick inhibitors (inhibitors of the tyrosine kinase zap-70 or Ick); inhibitors of vascular endothelial cell growth factor or vascular endothelial cell growth factor receptor as described herein; inhibitors of angiogenesis as described herein; corticosteroid anti-inflammatory drugs (e.g., SB203580); TNF-convertase inhibitors; IL-11 and IL-13 inhibitors; gold; penicillamine; chloroquine; hydroxychloroquine; chlorambucil; cyclophosphamide; cyclosporine; total lymphoid irradiation; antithymocyte globulin; CD5-toxins; orally administered peptides and collagen; lobenzarit disodium; cytokine regulating agents (CRAs) 11P228 and HP466 (Houghten Pharmaceuticals, Inc.); ICAM-1 antisense phosphorothioate oligodeoxucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline (MINOCIN®); anti-IL2R antibodies; marine and botanical lipids (fish and plant seed fatty acids); auranofin; phenylbutazone; meclofenamic acid; flufenamic acid; intravenous immune globulin; zileuton; mycophenolic acid RS-6 1443); tacrolimus (FK-506); sirolimus (rapamycin); amiprilose (therafectin); cladribine (2-chiorodeoxyadenosine); and azaribine.

Nonlimiting examples of agents for treating or preventing multiple sclerosis with which IL-17 modulators can be combined include the following: interferons, e.g., interferon-αla (e.g., AVONEX; Biogen) and interferon-1b (BETASERON Chiron/Berlex); Copolymer-1 (Cop-1; COPAXONE1′, Teva Pharmaceutical Industries, Inc.); hyperbaric oxygen; intravenous immunoglobulin; cladribine; TNF antagonists as described herein; corticosteroids; prednisolone; methyiprednisolone; azathiopine; cyclophosphamide; cyclosporine; cyclosporine A, methotrexate; 4-aminopyridine; and tizanidine. Additional antagonists include antibodies to or antagonists of other human cytokines or growth factors, for example, TNF, LT, IL-1, IL-2, IL-6, IL-7, IL-8, IL-12 IL-15, IL-16, IL-18 , IEMAP-11, GM-CSF, FGF, and PDGF.

Nonlimiting examples of agents for treating or preventing inflammatory bowel disease (e.g., Crohn's disease, ulcerative colitis) with which a IL-17 modulator can be combined include the following: budenoside; epidermal growth factor; corticosteroids; cyclosporine; sulfasalazine; amino salicylates; 6-mercaptopurine; azathiopine; metronidazole; lipoxygenase inhibitors; mesalamine; olsalazine; balsalazide; antioxidants; thromboxane inhibitors; IL-1 receptor antagonists; anti-IL-1 antibodies; anti-IL-6 antibodies; growth factors; elastase inhibitors; pyridinyl-imidazole compounds; TNF antagonists as described herein; IL-4, IL-6, IL-13 and/or TG193 cytokines or agonists thereof (e.g., agonist antibodies); IL-1, glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP 10; T Cell Sciences, Inc.); slow-release mesalazine; methotrexate; antagonists of platelet activating factor PAF); ciprofloxacin; and lignocaine.

Nonlimiting examples of agents for treating or preventing immune responses with which an IL-17 modulator can be combined include the following: antibodies against other cell surface molecules, including CD25 (interleukin-2 receptor-a), CD11a (LFA-1), CD54 (ICAM-1), CD4, CD45, CD28/CTLA4 (CD8O (B7.1), e.g., CTLA4 Ig-abatacept (ORENCIA®)), ICOSL, ICOS and/or CD86 (B7.2).

In other embodiments, IL-17 modulators can be used as vaccine adjuvants against autoimmune disorders, inflammatory diseases, etc. The combination of adjuvants for treatment of these types of disorders are suitable for use in combination with a wide variety of antigens from targeted self-antigens, i.e., autoantigens, involved in autoimmunity, e.g., myelin basic protein; inflammatory self-antigens, e.g. , amyloid peptide protein, or transplant antigens, e.g., alloantigens. The antigen may comprise peptides or polypeptides derived from proteins, as well as fragments of any of the following: saccharides, proteins, polynucleotides or oligonucleotides, autoantigens, amyloid peptide protein, transplant antigens, allergens, or other macromolecular components. In some instances, more than one antigen is included in the antigenic composition. For example, desirable vaccines for moderating responses to allergens in a vertebrate host, which contain the adjuvant combinations of this invention, include those containing an allergen or fragment thereof. Examples of such allergens are described in U.S. Pat. No. 5,830,877 and published International Patent Application No. WO 99/51259, which are incorporated herein by reference, and include pollen, insect venoms, animal dander, fungal spores and drugs (such as penicillin). The vaccines interfere with the production of IgE antibodies, a known cause of allergic reactions. In another example, desirable vaccines for preventing or treating disease characterized by amyloid deposition in a vertebrate host, which contain adjuvant combinations, include those containing portions of amyloid peptide protein (APP). This disease is referred to variously as Alzheimer's disease, amyloidosis or amyloidogenic disease. Thus, vaccines include the adjuvant combinations plus Aβ peptide, as well as fragments of A peptide and antibodies to Aβ peptide or fragments thereof.

C. Formulation and Administration

The present invention includes pharmaceutical compositions, which may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, intraocular, epidermal and transdermal), oral, via a medical device or parenteral. Parenteral administration includes intravenous, intraarterial, subcutaneous, intraocular, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, without limitation, penetration enhancers, carrier compounds and other pharmaceutically acceptable carriers or excipients.

Pharmaceutical compositions include, without limitation, solutions, emulsions, and liposome-containing formulations. These compositions may be generated from a variety of components that include, without limitation, preformed liquids, self-emulsifying solids and self-emulsifying semisolids.

Pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

Compositions may be formulated into any of many possible dosage forms such as, without limitation, tablets, capsules, liquid syrups, soft gels, suppositories, and enemas. Compositions may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment, pharmaceutical compositions may be formulated and used as foams. Pharmaceutical foams include formulations such as, without limitation, emulsions, microemulsions, creams, jellies and liposomes. While basically similar in nature these formulations vary in the components and the consistency of the final product.

If a compound includes an oligonucleotide, agents that enhance uptake of oligonucleotides at the cellular level may also be added to the pharmaceutical and other compositions. For example, cationic lipids, such as lipofectin (U.S. Pat. No. 5,705,188), cationic glycerol derivatives, and polycationic molecules, such as polylysine (PCT International publication No. WO 97/30731), also enhance the cellular uptake of oligonucleotides.

Compositions may additionally contain other adjunct components conventionally found in pharmaceutical compositions. Thus, for example, compositions may contain additional, compatible, pharmaceutically-active materials such as, for example, antipruritics, astringents, local anesthetics or anti-inflammatory agents, or may contain additional materials useful in physically formulating various dosage forms of the compositions, such as dyes, flavoring agents, preservatives, antioxidants, opacifiers, thickening agents and stabilizers. However, such materials, when added, should not unduly interfere with the biological activities of the components of the compositions. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. In one embodiment, oral formulations are those in which oligonucleotides of the invention are administered in conjunction with one or more penetration enhancers, surfactants and chelators. In other embodiments, surfactants include fatty acids and/or esters or salts thereof, bile acids and/or salts thereof. Bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). In addition, combinations of penetration enhancers, for example, fatty acids/salts in combination with bile acids/salts can be used. In one embodiment, the combination is the sodium salt of lauric acid, capric acid and UDCA. Further penetration enhancers include polyoxyethylene-9-lauryl ether, polyoxyethylene-20-cetyl ether. Oligonucleotides may be delivered orally in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. In one embodiment, complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

Other acceptable carriers or diluents are well known in the pharmaceutical art and are described, for example, in Remington's Pharmaceutical Sciences, Mack Publishing Co. IA.R. (Gennaro, ed., 1995).

When more than one compound or agent is administered, additional agents may be administered separately from the compound-containing composition, as part of a multiple dosage regimen. Alternatively, those agents may be part of a single dosage form, mixed together with the compound of this invention in a single composition. If administered as part of a multiple dosage regime, the two active agents may be submitted simultaneously, sequentially or within a period of time from one another normally within five hours from one another.

The amount of both the compound and the additional therapeutic agent (in those compositions which comprise an additional therapeutic agent as described above) that may be combined with the carrier materials to produce a single dosage form will vary depending upon the host treated and the particular mode of administration.

### IV. Kits

Another aspect of the present invention relates to kits for preparing TH-17 cells. In one embodiment, the kit comprises one or more cytokines for inducing IL-17 expression in T-cells, as well as one or more antibodies. A kit can further comprise molecules for measuring IL-17 expression, including, without limitation, IL-17 antibodies and/or oligonucleotides or polynucleotides that hybridize with IL-17 mRNA. In another aspect, the invention relates to kits for identifying modulators of IL-17. A kit can comprise one or more cytokines for inducing IL-17 expression in T-cells as well as one or more antibodies. A kit can further comprise an agent that modulates IL-17 expression in T-cells. In a further embodiment, a kit can further comprise molecules for measuring IL-17 expression, including, without limitation, IL-17 antibodies and/or oligonucleotides or polynucleotides that hybridize with IL-17 mRNA.

The following examples are offered for illustrative purposes only, and are not intended to limit the scope of the present invention in any way.

### V. Examples

### Example 1

### In Vitro Polarization of TH-17 Cells

Peripheral Blood Mononuclear Cells (PBMCs) were isolated from the buffy coat of human peripheral blood and aliquoted into 12-well culture plates coated with anti-human CD3. Lipopolysaccharide (LPS) was added to a final concentration of 50 ηg/mL, human TGFβ to a final concentration of 10 ηg/mL, and anti-human IFNγ, anti-human IL-4 and anti human IL-12 were added to a final concentration of 10 μg/mL each. Anti-human CD28 was added as a co-stimulus to a final concentration of 1 μg/mL. The cells were incubated at 37° C. for 2 days. Human IL-2 was added to a final concentration of 2 ηg/mL and the cells were incubated for three more days.

In a second method, naïve T-cells were purified from PBMCs by depletion of non-T-helper cells and memory T-helper cells (negative selection). Non-T-helper cells and memory T-helper cells are indirectly magnetically labeled with a cocktail of biotin-conjugated monoclonal antibodies, as primary labeling reagent, and anti-biotin monoclonal antibodies conjugated to Microbeads, as secondary labeling reagent. The magnetically labeled non-T-helper cells and memory T-helper cells are depleted by retaining them on a MACS Column in the magnetic field of a MACS Separator, while the unlabelled naïve T helper cells pass through the column. Naïve T-cells were incubated in conditioned media in the presence of a T-cell receptor (TCR)/CD-28 T-cell stimulus. Conditioned media was taken from LPS (50 ηg/m1)-stimulated PBMCs incubated for 24 hours in complete media (RPMI, 10% FCS, 1% PS, 1% MEM Vitamins, 10 μg/mL insulin, 5.5 μg/ml Transferrin, 6.7 ηg/mL Selenium and 50 μM β-mercaptoethanol. LPS was added as a pro-inflammatory dendritic cell (DC) stimulus. Exogenous human IL-6 (20 ηg/ml), human TGFβ (10 ηg/ml), human IL-23 (200 ηg/ml) and blocking antibodies for IL-4, IL-12 and IFNγ (10 μg/ml) were also added to ensure blockade of TH1 and TH2 differentiation. Cells were incubated for 5 days prior to characterization.

### Example 2

### Characterization In Vitro Polarised of Th-17 Cells

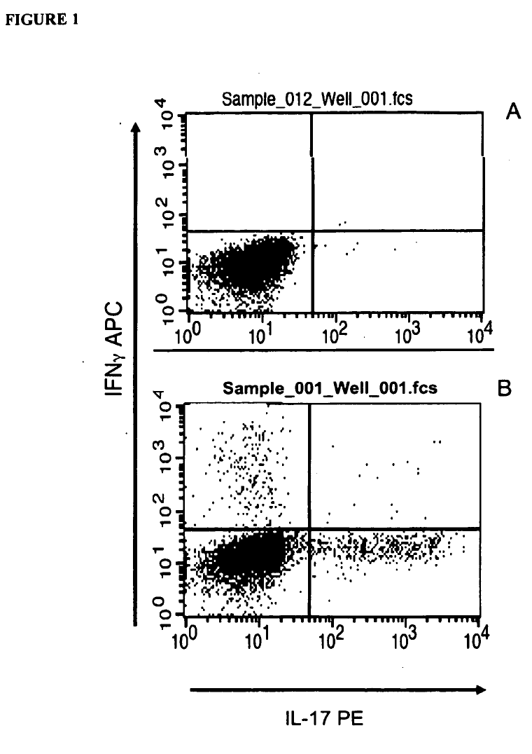

T cells that had previously been stimulated under TH-17-polarising conditions were resuspended at 1×10⁶/mL and left unstimulated or stimulated with PMA (50 ηg/mL) and lonomycin (250 ηg/mL) for 5 hours. Brefeldin-A was added for the final 4 hours to block protein transport to the Golgi complex and accumulate proteins in the endoplasmic reticulum. A cocktail of conjugated antibodies (IL-17-PE and IFNγ-APC) were used to characterize the cells by FACS analysis. The results of FACS analysis are shown in FIG. 1, which shows higher levels of IL-17 and IFNγ in stimulated TH-17 cells.

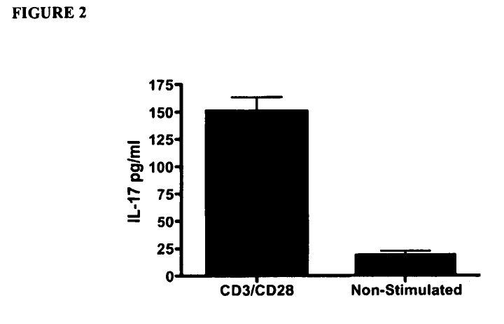

TH-17 cells were further characterized by quantifying the amount of IL-17 produced by the cells. TH-17 cells were resuspended at 4×10⁶/mL and incubated in the presence or absence of a T-cell receptor (TCR)/CD-28 T-cell stimulus for 24 hours at 37° C. The levels of IL-17 within the supernatants were quantified using a fluorescent bead immunoassay kit. Results in FIG. 2 show more than 10-fold higher levels of IL-17 from TH-17 cells stimulated with a (TCR)/CD-28 T-cell stimulus compared with unstimulated TH-17 cells.

### Example 3

### Memory CD4 T-Cell IL-17 Assay

Detectable levels of IL-17 are produced in anti-CD3/anti-CD28 co-stimulated human PBMC preparations. FACs analysis revealed that the vast majority of IL-17 producing PBMC cells have a TH-17 phenotype, i.e., they are CD3 positive, CD4 positive, CD45RO positive (i.e., memory T cells) IL-17 positive and IFNγ negative.

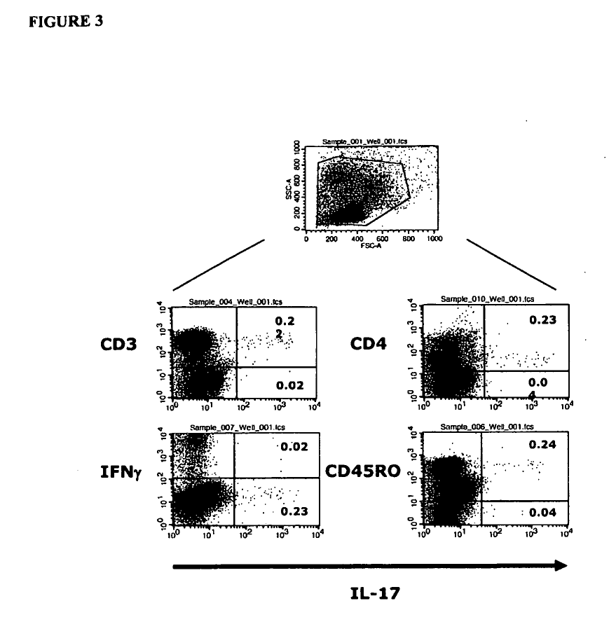

FIG. 3 shows that IL-17 secreting cells in human PBMC preparations have a TH-17 cell phenotype. Human PBMCs were stimulated with PMA and ionomycin for 6 hours. Cells were stained with fluorescently labeled antibodies against CD3, CD4 IFNγ (BD Biosciences) and IL-17 (EBioscience) and analysed by FACs.

The observation that TH-17 cells are already present in human PBMC preparations provides a way to measure TH-17 cell function without having to conduct time consuming and expensive in vitro polarization of naïve human T cells into a TH-17 phenotype. In the memory CD4 T cell IL-17 assay, TH-17 cells are enriched by purifying memory T cells (CD4 positive, CD45RO positive) from human PBMC preparations and the effect of test compounds on IL-17 production after anti-CD3/anti-CD28 co-stimulation is assessed. First, memory T cells are purified from human PBMCs on a MACs column, using a human memory T cell negative selection kit (Miltenyi Biotech). Second, purified memory T cells are re-suspended in complete media at a cell density of 5×10⁵ cells/mL. Third, memory T cells, 100 μwell, are dispensed into anti-CD3 coated 96-well plates, containing anti-CD28 and test compound (or agent) or vehicle control. Fourth, plates are incubated for 24 h at 37° C., 5% CO₂. Fifth, supernatants are collected and IL-17 levels determined by either ELISA (R&D systems) or BioPlex assay (Bio-Rad Laboratories).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages and modifications are within the scope of the following claims.

All patent and literature references cited in the present specification are incorporated herein by reference in their entirety.

What is claimed is:

1. A method of generating IL-17 producing T-cells comprising
a) obtaining naïve T-cells from a mammal from any order except rodentia;
b) exposing the T-cells to conditioned media from LPS-stimulated PBMCs;
c) exposing the T-cells to a T-cell receptor/CD28 T-cell stimulus;
d) exposing the T-cells to LPS
e) exposing the T-cells to TGFβ;
f) exposing the T-cells to one or more of anti IFNγ, anti IL-4, and IL-12; and
g) exposing the T-cells to IL-6.

2. The method of claim 5, wherein the T-cells are exposed to anti IFNγ, anti IL-4, and IL-12.

3. The method of claim 5, wherein the mammal is a primate.

4. The method of claim 5, wherein the primate is a human.

5. A method of generating IL-17 producing T-cells comprising
a) obtaining peripheral blood mononucleocytes (PBMCs) from a mammal from any order except rodentia;
b) exposing the PBMCs to anti CD3;
c) exposing the PBMCs to Lipopolysaccharide (LPS);
d) exposing the PBMCs to TGFβ;
e) exposing the PBMCs to one or more of anti IFNγ, anti IL-4, and IL-12;
f) exposing the PBMCs to anti CD28; and
g) exposing the PBMCs to IL-2.

6. The method of claim 1, wherein the PBMCs are exposed to anti IFNγ, anti IL-4, and IL-12.

7. The method of claim 1, wherein the mammal is a primate.

8. The method of claim 3, wherein the primate is a human.

9. An isolated human IL-17 producing T-cell.

10. An isolated human IL-17 producing T-cell, wherein the cell was produced by the method of claim 1 or claim 2.

11. An isolated human IL-17 producing T-cell, wherein the cell was produced by the method of claim 5 or claim 6.

12. A method for identifying a modulator of IL-17 production in T-cells comprising:
a) contacting cells from a mammal, wherein the cells are capable of producing IL-17, with a candidate agent and
b) measuring the amount of IL-17 produced by the cells.

13. A method for identifying a modulator of IL-17 production in T-cells comprising:
a) obtaining naïve T-cells from a mammal;
b) exposing the T-cells to conditioned media from LPS-stimulated PBMCs;
c) exposing the T-cells to a T-cell receptor/CD28 T-cell stimulus;
d) exposing the T-cells to LPS, TGFβ and to one or more of anti IFNγ, anti IL-4, and IL-12;
e) contacting the cells with a candidate agent;
f) exposing the T-cells to IL-6; and
g) measuring the amount of IL-17 produced by the cells.

14. A method for identifying a modulator of IL-17 production in T-cells comprising:
a) obtaining peripheral blood mononucleocytes (PBMCs) from a mammal;
b) exposing the PBMCs to anti CD3;
c) exposing the PBMCs to lipopolysaccharide (LPS) and TGFβ;
d) exposing the PBMCs to one or more of anti IFNγ, anti IL-4, and IL-12;
e) exposing the PBMCs to anti CD28;
f) contacting the cells with a candidate agent;
g) exposing the PBMCs to IL-2; and
h) measuring the amount of IL-17 produced by the cells.

15. A method for identifying a modulator of IL-17 production in T-cells comprising:
a) obtaining peripheral blood mononucleocytes (PBMCs) from a mammal;
b) purifying memory T-cells from PBMCs;
c) exposing the memory T-cells to anti CD3;
d) exposing the memory T-cells to anti-CD28
e) contacting the cells with a candidate agent; and
f) measuring the amount of IL-17 produced by the cells.

16. The method of any of claims 12-15, wherein the cells are from a human.

17. The method of any of claims 12-16, wherein the amount of IL-17 is measured by ELISA.

18. The method of claim 15, wherein the memory T-cells are purified using negative selection.

19. The method of any of claims 12-18, wherein the candidate agent is a small molecule.

20. The method of any of claims 12-18, wherein the candidate agent is a non-peptide small organic molecule.

21. The method of any of claims 12-18, wherein the candidate agent is an oligonucleotide.

22. The method of any of claims 12-18, wherein the candidate agent is a peptide.

23. The method of any of claims 12-18, wherein the candidate agent is a polypeptide.

24. The method of any of claims 12-18, wherein the candidate agent is an antibody or a fragment thereof.

25. The method of any of claims 12-24, further comprising the step of identifying the candidate agent as a modulator of IL-17 production if the amount of IL-17 is higher or lower in the presence as compared to the absence of the candidate molecule.

26. A modulator of IL-17 production identified by the method of any of claims 12-25.

27. A method for modulating interleukin-17 (IL-17) production by T-cells comprising treating T-cells with a modulator of IL-17 production.

28. The method of claim 27, wherein the modulator is identified by any of claims 12-25.

29. The method of claim 27 or claim 28, wherein the modulator decreases IL-17 production.

30. The method of claim 27 or 28, wherein the modulator increases IL-17 production.

31. A method of treating an IL-17-mediated disorder in a cell or mammal, comprising administering to a cell or mammal an effective amount of a modulator of IL-17 production in T-cells.

32. The method of claim 31, wherein the modulator is identified by any of claims 12-25.

33. The method of claim 31 or claim 32, wherein the IL-17 mediated disorder is multiple sclerosis.

34. The method of claim 31 or claim 32, wherein the IL-17 mediated disorder is rheumatoid arthritis.

35. The method of claim 31 or claim 32, wherein the IL-17 mediated disorder is psoriasis.
